(19) United States
(12) Patent Application Publication
(10) Pub. No.: US 2009/0149879 A1
(43) Pub. Date: Jun. 11, 2009

(54) Dynamic volume displacement weight loss device

(76) Inventor: Travis E. Dillon, Winston-Salem, NC (US)

Correspondence Address: BRINKS HOFER GILSON & LIONE/CHICAGO/COOK, PO BOX 10395, CHICAGO, IL, 60610, US

(21) Appl. No.: 11/953,720

(22) Filed: Dec. 10, 2007

(51) Int. Cl. A61M 29/02

(52) U.S. Cl. 606/192

(57) ABSTRACT

An intragastric device and method of use thereof are provided. The device is actuated to change its volume based on one or parameters detected in the gastric lumen. The device comprises an expandable reservoir that is adapted to distend one or more walls of the gastric lumen for a predetermined time. The device may also be actuated based on a pressure control system in which the reservoir maintains a constant pressure against the walls of the gastric lumen.

### TECHNICAL FIELD

This invention relates to medical devices, and more particularly to obesity treatment devices.

### BACKGROUND OF THE INVENTION

It is well known that obesity is a very difficult condition to treat. Methods of treatment are varied, and include drugs, behavior therapy, and physical exercise, or often a combinational approach involving two or more of these methods. Unfortunately, results are seldom long term, with many patients eventually returning to their original weight over time. For that reason, obesity, particularly morbid obesity, is often considered an incurable condition. More invasive approaches have been available which have yielded good results in many patients. These include surgical options such as bypass operations or gastroplasty. However, these procedures carry high risks, and are therefore not appropriate for most patients.

In the early 1980s, physicians began to experiment with the placement of intragastric balloons to reduce the size of the stomach reservoir, and consequently its capacity for food. Once deployed in the stomach, the balloon helps to trigger a sensation of fullness and a decreased feeling of hunger. These balloons are typically cylindrical or pear-shaped, generally range in size from 200-500 ml or more, are made of an elastomer such as silicone, polyurethane, or latex, and are filled with air, water, or saline. While some studies demonstrated modest weight loss, the effects of these balloons often diminished after three or four weeks, possibly due to the gradual distension of the stomach or the fact that the body adjusted to the presence of the balloon. Other balloons include a tube exiting the nasal passage that allows the balloon to be periodically deflated and re-insufflated to better simulate normal food intake. However, the disadvantages of having an inflation tube exiting the nose are obvious.

The experience with volume displacing, weight loss devices (VDWLD's), such as intragastric balloons as a method of treating obesity have provided uncertain results, and have been frequently disappointing. Some trials failed to show significant weight loss over a placebo, or were ineffective unless the balloon placement procedure was combined with a low-calorie diet. Complications have also been observed, such as gastric ulcers, especially with use of fluid-filled balloons, and small bowel obstructions caused by deflated balloons. In addition, there have been documented instances of the balloon blocking off or lodging in the opening to the duodenum, wherein the balloon may act like a ball valve to prevent the stomach contents from emptying into the intestines.

Additionally, intragastric balloons are intended to displace a fixed volume after they have been implanted in the stomach. A problem with current intragastric balloons is that they chronically distend the stomach walls. These intragastric balloons are not based on a specific patient's threshold of satiety and discomfort level. Rather, the intragastric balloon is inflated to a predetermined volume based on the patient's stomach size. Because the volume of the balloon remains fixed, the balloon is constantly exerting a force against the walls of the stomach. This can lead to vomiting and nausea as the patient tries to adjust to the intragastric balloon.

Moreover, the stomach may eventually adjust to the balloon by increasing in size. The balloon at this point must be removed because the patient has outgrown it. Upon removal of the balloon, the stomach has actually become larger in size such that the patient can eat more.

In view of the drawbacks of current intragastric devices, there is an unmet need for an improved intragastric device that substantially eliminates the adverse effects associated with displacing a fixed volume in the stomach.

### SUMMARY OF THE INVENTION

Accordingly, an intragastric device is provided that is actuated to change volume in response to one or more detected parameters after being implanted in the gastric lumen. Although the inventions described below may be useful for substantially eliminating the adverse effects associated with disposing a fixed volume intragastric device in the stomach, the claimed inventions may also solve other problems.

In a first aspect, an intragastric device for the treatment of obesity is provided. A reservoir is provided that comprises an elastic material that is configured to change volume while implanted within a gastric lumen. The reservoir is actuated to change volume in response to one or more detected parameters, and the reservoir is adapted to distend one or more walls of the gastric lumen for a predetermined time.

In a second aspect, an intragastric device for the treatment of obesity is provided. The intragastric device comprises an expandable reservoir that is configured to change volume while implanted in a gastric lumen. The reservoir is actuated by a pressure controller to change volume in response to a pressure being exerted against the reservoir. The reservoir is adapted to distend one or more walls of the gastric lumen for a predetermined time to trigger a sensation of satiety.

In a third aspect, a method of treatment of obesity is provided. A reservoir is introduced into a gastric lumen in which the reservoir has a first volume. A parameter is detected within the gastric lumen, the parameter being indicative of expansion of the gastric lumen. The reservoir is actuated based on the detected parameter such that the reservoir changes from the first volume to a second volume, the second volume being larger than the first volume. The reservoir engages a wall of the gastric lumen to distend the wall of the gastric lumen for a predetermined time.

### BRIEF DESCRIPTION OF SEVERAL VIEWS OF THE DRAWINGS

FIG. 1 shows a reservoir engaging and distending an upper portion of the stomach;

FIG. 2 shows the reservoir of FIG. 1 in a non-distended state;

FIG. 3 shows another embodiment in which two reservoirs are interconnected by a micro-pump;

FIG. 4 shows another embodiment in which an external pump forces air through a percutaneous tube to inflate a reservoir;

FIG. 5 shows yet another embodiment in which a tube extends from the reservoir and pump through the esophagus and nose of a patient;

FIGS. 6 and 7 show yet another embodiment of a graph indicating the operation of a pressure actuated reservoir; and

FIG. 8 shows an example of a pressure actuated reservoir.

### DETAILED DESCRIPTION OF THE INVENTION

The embodiments are described with reference to the drawings in which like elements are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not to scale and in certain instances details have been omitted, which are not necessary for an understanding of the embodiments, such as conventional details of fabrication and assembly.

The term “fluid” as used herein refers to any type of biocompatible fluid, air, or gas that is suitable for being introduced into the intragastric device. The term “distended” as used herein refers to a configuration of the intragastric device within the gastric lumen that induces a sensation of satiety.

Various intragastric devices to treat obesity will be discussed that are capable of changing volume while implanted in a gastric lumen. The devices may be actuated to increase and decrease in volume based on a patient's specific satiety perception and threshold of discomfort (FIGS. 1-5). The volume actuation may be based on variety of parameters, such as the pH of the gastric lumen, the temperature of the gastric lumen, or predetermined time intervals. The devices are designed to increase to a specific patient's predetermined satiety inducing volume such that the sensation of satiety can be achieved. At the same time, the devices are designed to not exceed a predetermined volume so that adverse effects such as substantial vomiting and nausea do not occur.

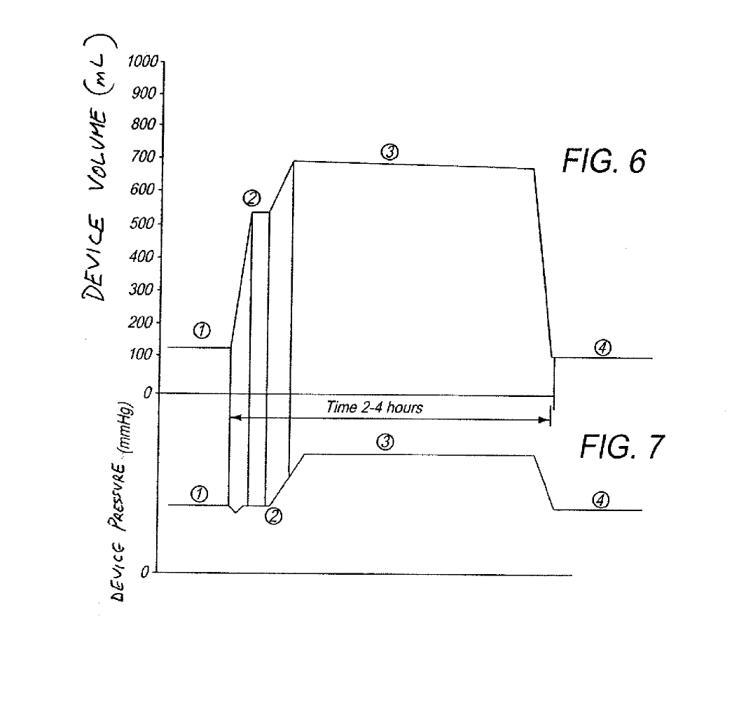

Alternatively, the devices may be actuated on the basis of a predetermined distension pressure which triggers a patient specific satiety level (FIGS. 6-7). The term “distension pressure” as used herein is intended to mean the pressure exerted by the device against a gastric wall. At the same time, the pressure-actuated devices are designed to not exceed a patient specific satiety pressure (i.e., the pressure at which a particular individual will have the sensation of feeling full) so that adverse effects such as substantial vomiting and nausea do not occur.

It should be noted that the present invention is not limited to any of the embodiments that will be described herein. Rather, the embodiments are intended to serve illustrative purposes only.

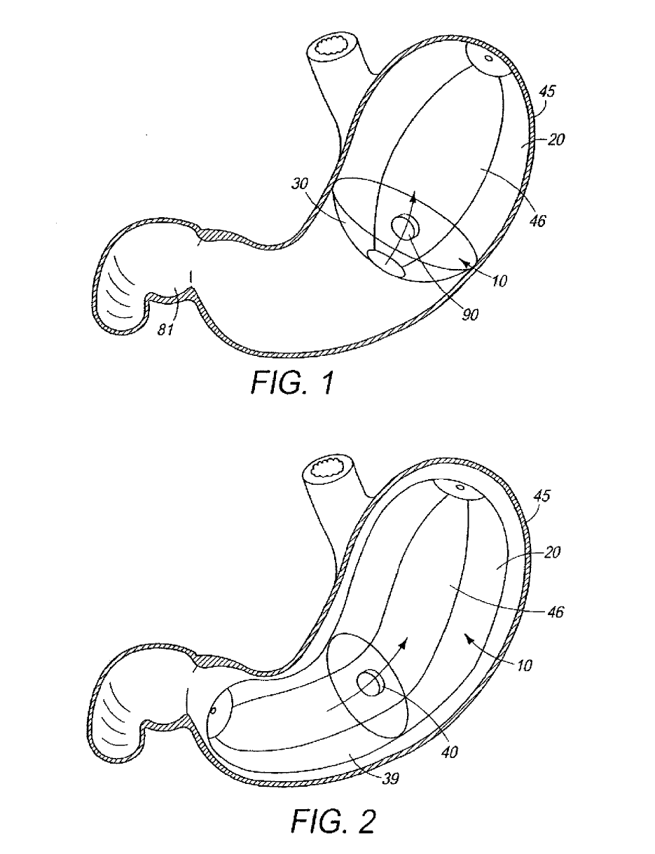

FIGS. 1 and 2 show an embodiment of an enclosed reservoir 10 comprising a top portion 20 and a bottom portion 30. The top portion 20 and the bottom portion 30 are in fluid communication with each other by a valve 40. Fluid may be exchanged back and forth between the top and the bottom portions 20 and 30 through the valve 40 to alter their respective volumes such that the top and the bottom portions 20 and 30 are transitionable between a non-distended state and a distended state. In the example of FIG. 1, all of the fluid from the bottom portion 30 has traveled through valve 40 into the top portion 20 such that the top portion 20 comprises a volume of about 1000 mL and the bottom portion 30 comprises about zero volume. The valve 40 is closed off to maintain the fluid in the top portion 20. In this example, because the top portion 20 occupies a sufficient volume of about 1000 mL, the top portion 20 engages the upper walls 45 of the gastric lumen 46. The engagement of the top portion 20 with the walls 45 exerts a sufficient force therealong to distend the upper walls 45 of the gastric lumen 46 and thus induce the feeling of satiety.

The reservoir 10 possesses the capability to transition between the distended state of FIG. 1 and the non-distended state of FIG. 2, as will now be discussed. The valve 40 may be opened such that fluid travels out of top portion 20 and into the bottom portion 30, as shown by the arrows in FIG. 1, through the valve 40. A pump, located either externally or internally within the gastric lumen 46, may be used to direct the fluid through the valve 40. As fluid passes through the valve 40, the fluid exerts a pressure on the bottom surface of the reservoir 10 thereby causing the bottom portion 30 of the reservoir 10 to increase in size such that it takes the shape shown in FIG. 2. The bottom portion 30 of the reservoir 10 stretches downward toward the bottom portion of the gastric lumen 46 (i.e., the antrum). The result is that the bottom portion 30 of the reservoir 10 increases in volume from about zero volume to about 300 mL, and the top portion 20 proportionally decreases in volume from about 1000 mL to about 700 mL. Accordingly, the overall volume of the reservoir 10 remains constant at about 1000 mL, but the overall shape of the reservoir 10 changes configuration to a non-distended state. In particular, FIG. 2 shows that the reservoir 10 has a configuration that is more stretched out in the gastric lumen 46 than the configuration of the reservoir 10 shown in FIG. 1. FIG. 2 indicates that the reservoir 10 is not engaging any wall 45 of the gastric lumen 46. Accordingly, none of the walls of the gastric lumen 46 are distended to induce satiety. The reservoir 10 in its non-distended state possesses sufficient volume such that it does not migrate into the pylorus 81.

Referring to FIG. 2, fluid may flow upwards through the valve 40, as indicated by the arrow, to re-establish the reservoir 10 distended state configuration of FIG. 1. The transitioning of the reservoir 10 to a distended state may occur before food intake or during food intake. Preferably, the transitioning of the reservoir 10 to a distended state occurs during food intake so that the patient can receive some nutrients. During the transitioning of the reservoir 10 to a distended state, the food particles move around the top and bottom portions 20 and 30.

Unlike conventional intragastric balloons which chronically distend the stomach walls; the reservoir 10 has the ability to constantly transition between a distended state and a non-distended state in accordance with a patient's perception of satiety. As an example, temperature and/or pH sensors may be connected to a microcontroller to detect when the transitioning between distended and non-distended states will occur, as will be discussed in greater detail below. Alternatively, the microcontroller may be programmed at particular time intervals (e.g., every day at noon when the person consumes food) to direct the pump to move fluid through the valve 40 so as to create a distended state.

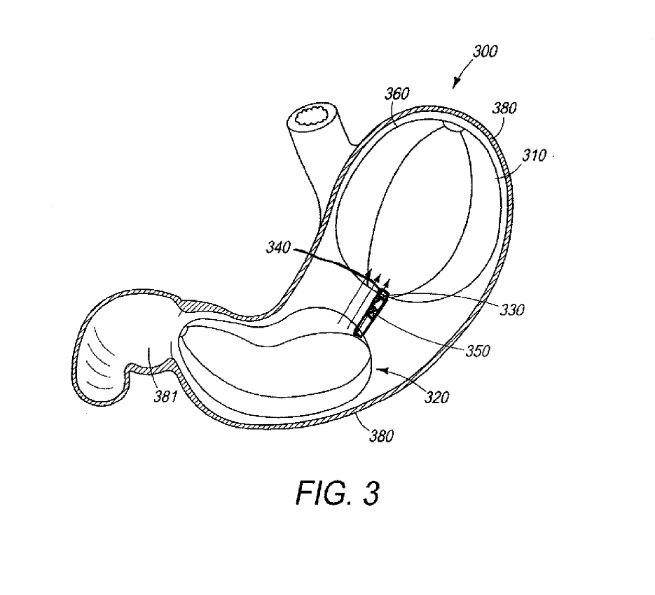

FIG. 3 is another example of a dynamic volume actuation system 300 to induce satiety for a predetermined period of time. The dynamic volume actuation system 300 is a closed system that comprises a top reservoir 310, a bottom reservoir 320, a pump 330, a valve 350, and a microcontroller 340. The top and bottom reservoirs 310 and 320 are in fluid communication with each other by the pump 330 and the microcontroller 340, which act as a membrane between the reservoirs 310 and 320. The pump 330 directs fluid between the top and bottom reservoirs 310 and 320 when a microcontroller 340 senses food intake on the basis of one or more parameters (e.g., a rise in pH level and/or drop in temperature within the gastric lumen). Generally speaking, any parameter which signals the stomach to be relaxing can be a parameter that the microcontroller 340 senses and uses as a basis to actuate movement of fluid between the bottom reservoir 320 and the top reservoir 310 for the purpose of expanding and deflating the top reservoir 310 to distend and non-distend the walls 380 of the upper gastric lumen 360.

Electrical leads may be implanted within the gastric lumen 360 that detect one or more of these parameters. One end of each of the electrical leads is then connected to the microcontroller 340. The microcontroller 340 is in electronic communication with the pump 330 and the valve 350.

In the example of FIG. 3, the microcontroller's 340 detection of one or more changed parameters to detect food intake triggers actuation of the pump 330. The pump 330 directs fluid from the bottom reservoir 320 to the top reservoir 310. A predetermined amount of fluid travels from the bottom reservoir 320 to the top reservoir 310 through the valve 350 as indicated by upward arrows 370. As a result of the fluid movement, the bottom reservoir 320 decreases in volume and the top reservoir 310 proportionally increases in volume. The top reservoir 310 increases to a sufficient volume to distend the walls 380 of the upper gastric lumen 360 such that satiety is induced for a predetermined amount of time.

At this juncture, the microcontroller 340 senses that satiety has been achieved at the upper portion of the gastric walls 380. Detection of satiety by the microcontroller 340 causes it to transmit a signal to the pump 330. The signal deactivates the pump 330 such that the pump 330 stops pumping fluid from the bottom reservoir 320 to the top reservoir 310. Valve 350 closes off to ensure that fluid remains in the top reservoir 310 and does not flow back into the bottom reservoir 320. The increase in volume of the top reservoir 310 is sufficient to engage and distend the upper walls 380 of the gastric lumen 360. The time period of distension is patient specific. Preferably, the time period of distension is sufficient to allow the food particles to digest and exit through the pylorus 381 so as to prevent the patient from immediately consuming food.

The microcontroller 340 detects when the food particles have exited the gastric lumen 360. The microcontroller 340 can detect the exit of food particles from the gastric lumen 360 in a number of ways. In one example, the microcontroller 340 may be programmed to a predetermined time duration which is equal to the time required for a particular person to empty food contents from their gastric lumen 360. Such a predetermined time duration can be determined experimentally and is patient specific. Alternatively, the microcontroller 340 may detect when the food particles have exited the gastric lumen 360 by sensing when peristalsis has occurred. The microcontroller 340 may sense a series of pressure spikes over time as the gastric lumen 360 undergoes multiple wavelike contractions to force food contents out of the gastric lumen 360 and into the pylorus 381 and duodenum. The microcontroller 340 monitors the series of pressure spikes over time and can determine when the contractions have ended, which indicates that the food contents have emptied from the patient's gastric lumen 360.

After the microcontroller 340 has detected that the food contents have exited the gastric lumen 360 and passed through the pylorus 381 and into the duodenum, the microcontroller 340 transmits a signal to the pump 330 to return fluid from the top reservoir 310 to the bottom reservoir 320. The valve 350 opens for fluid to travel therethrough. The configuration of FIG. 2 is re-established in which both reservoirs 310 and 320 are in a nondistended state. While in the nondistended state, neither of the reservoirs 310 and 320 may engage the walls 380 of the gastric lumen 360. Thus, the dynamic volume actuation system 300 cycles between a nondistended state and a distended state depending on whether food intake is detected. The ability of the system 300 to selectively cycle between the two states may substantially eliminate discomfort levels of the patient due to chronic distension.

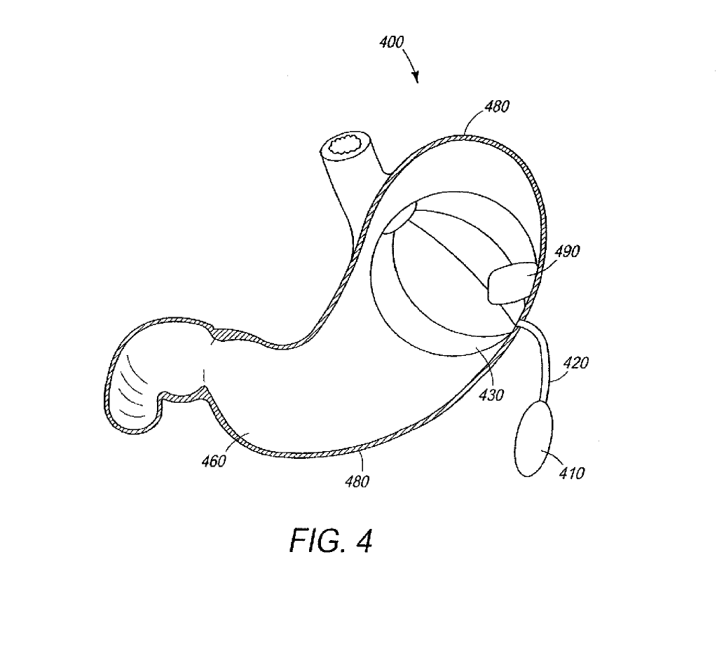

FIG. 4 is yet another example of a dynamic volume actuation system 400. Unlike the closed systems described in FIGS. 1-3 in which fluid moves between two reservoirs, FIG. 4 shows a dynamic volume actuation system 400 in which a single expandable intragastric balloon 430 inflates and deflates to change volume in response to one or more suitable parameters detected by a microcontroller 490. The system 400 comprises a pump 410, a percutaneous tube 420, a microcontroller 490, and an expandable intragastric balloon 430. The system 400 of FIG. 4 is an open system in which fluid (e.g., air) from the outside ambient atmosphere is used to inflate the balloon 430. The microcontroller 490 may be placed within or outside the gastric lumen 460. The percutaneous tube 420 is the conduit for the air, and it connects the balloon 430 to the pump 410. Generally speaking, the walls 480 of the gastric lumen 460 are distended to a satiety induced volume by pumping outside air through the tube 420 using the pump 410. The air travels through the percutaneous tube 420 and into the balloon 430, thereby causing the balloon 430 to inflate. When the microcontroller 490 senses that satiety has been achieved, it transmits a signal to the pump 410. The signal deactivates the pump 410 such that the pump 410 stops pumping air from the outside ambient atmosphere into the balloon 430. A valve closes off to ensure that the air does not leak out from balloon 430. The increase in volume of the balloon 430 is sufficient to engage and distend the wall of the gastric lumen 460.

After the food particles have digested and exited the pylorus, the balloon 430 may reduce in volume such that it no longer is distending the wall of the gastric lumen. The microcontroller 490 detects that the food particles have digested and exited the pylorus. Upon such detection, the microcontroller 490 transmits a signal to open the valve such that the pressurized air from the interior of the balloon 430 may exit through tube 420 and into the outside ambient atmosphere.

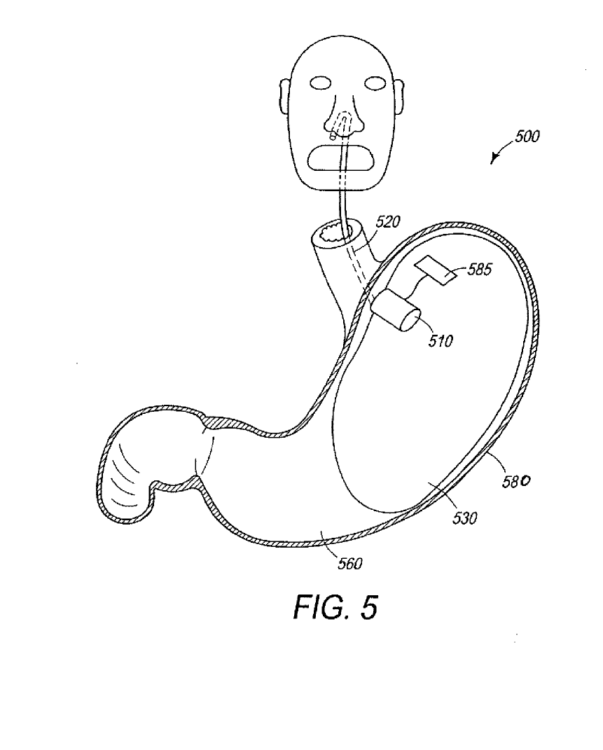

FIG. 5 is an alternative percutaneous dynamic volume actuation system 500. Rather than have the percutaneous tube 420 of FIG. 4 pass through the stomach wall and outside of the body, the percutaneous tube 520 of FIG. 5 is shown to extend along the esophagus and out of the nose of the patient. Additionally, a pump 510 is placed internally within the gastric lumen. The pump 510 is shown to be in electrical communication with a microcontroller 585.

Volume actuation of the above described dynamic systems may also be based on the pressure exerted by the walls of the gastric lumen against the reservoir. Pressure sensors or a strain gauge may be placed along the surface of the reservoir to detect the pressure being exerted by the walls of the gastric lumen along the surface of the reservoir. Alternatively, a pressure transducer may be positioned within the interior region of the reservoir that is capable of sensing changes in pressure. In another design, a diaphragm may be located at the pump 510 shown in FIG. 5 to sense the internal pressure of the reservoir.

Generally speaking, when the walls of the gastric lumen expand due to food intake, the pressure exerted by the reservoir against the walls decreases. The pressure sensors will detect such decrease in pressure and transmit a signal to a microcontroller. The microcontroller will then send instructions to a device (e.g., a pump) that enables the reservoir to expand such that the pressure increases and returns to its predetermined level, the predetermined level being known as the mean distension pressure (MDP). The MDP is defined as the lowest pressure level that provides a reservoir volume or intraballoon volume of 30 mL as known in the art. The MDP varies from patient to patient. During food intake into the gastric lumen, the microcontroller maintains the pressure exerted by the reservoir against the walls of the gastric lumen substantially constant at about the MDP level. Maintaining the reservoir at about the MDP level allows the microcontroller to monitor the changes in volume that the reservoir undergoes. When the microcontroller has sensed an increase in volume, it knows that food intake is occurring. After a predetermined time from which it has determined that food intake is occurring, the microcontroller relays a signal to the pump to turn on and increase the volume of the reservoir so as to create a patient specific satiety induced pressure, which is the pressure exerted by the reservoir against the walls of the gastric lumen to trigger a sensation of fullness. Similar to the MDP, the satiety induced pressure is patient specific and can be determined experimentally.

Prior to beginning the pressure-controlled procedure as shown in FIGS. 6 and 7, the MDP, satiety induced pressure level, and discomfort pressure level are determined for the particular patient. These parameters are patient specific. The MDP may be empirically determined by inserting a balloon into the proximal region of the stomach and increasing the pressure of the balloon in 1 mm Hg increments at a predetermined time interval (e.g., about every 3 minutes) until the volume of the balloon has increased to about 30 mL. The discomfort pressure level represents the pressure which, if exceeded, causes severe discomfort. These three parameters remain constant for a particular patient but vary from patient to patient. Generally speaking, according to published literature in the art, the average MDP is about 7 mm Hg and the average satiety pressure is about 12 mm Hg beyond the MDP. After obtaining these parameters, the pressure controlled actuation procedure may begin.

FIGS. 6 and 7 show a graph of the mechanism by which the pressure-controlled actuation procedure may occur. FIGS. 6 and 7 will be described in conjunction with the dynamic volume actuation system 300 described in FIG. 3. The vertical scale of FIG. 6 indicates the volume of the top reservoir 310 and the horizontal scale indicates time. The vertical scale of FIG. 7 represents the pressure exerted by the top reservoir 310 against the walls of the gastric lumen and the horizontal scale indicates time. It should be understood that the present invention is not limited to the specific volume and pressure values that will be described in FIGS. 6 and 7. Rather, the specific values are merely for illustration purposes of how the present invention operates.

Phase 1 (first segments of FIG. 6 and FIG. 7) represents the top reservoir 310 being configured in a non-distended state in which the pressure of the reservoir is held constant at about 2 mm Hg above the MDP to ensure that the reservoir 310 is engaging with the walls 380 of the gastric lumen 360. The top reservoir 310 at Phase 1 has a volume of about 120 mL that corresponds to the pressure in the reservoir of about 2 mm Hg above the MDP. This volume of the top reservoir 310 remains unchanged until the walls 380 of the gastric lumen 360 begin to relax and expand due to food intake. During Phase 1, the top reservoir 310 does not exert a satiety induced pressure. The top reservoir 310 at Phase 1 may possess the configuration as shown in FIG. 2.

When food intake occurs, the walls 380 of the gastric lumen 360 unfold and expand, thereby causing the top reservoir 310 to momentarily exert less pressure on the walls 360, as indicated by the slight dip and variable pressure level between Phases 1 and 2 in FIG. 7. The pressure sensors detect that the pressure exerted by the top reservoir 310 against the walls 380 of the gastric lumen 360 has momentarily decreased. In response to the decrease in reservoir 310 pressure, the pressure sensors transmit a first signal to the microcontroller 340 which in turn sends a second signal to a device such as the pump 330 to increase the volume of the top reservoir 310 so as to re-establish the about 2 mm Hg above the MDP, shown at Phase 2. Introduction of fluid from the bottom reservoir 320 into the top reservoir 310 enables the top reservoir 310 to expand until the pressure exerted by the top reservoir 310 against the walls 380 of the gastric lumen 360 has increased and returned to the original pressure level of about 2 mm Hg above the MDP as shown in Phase 2 of FIG. 7. The re-establishment of this pressure level can be seen in FIG. 7 as the variable pressure level segment between the plateaus of Phase 1 and Phase 2

At Phase 2, the top reservoir 310 has increased in volume to maintain the predetermined pressure level at about 2 mm Hg above the MDP. In this example, the pump 330 has introduced about 430 mL of fluid into the top reservoir 310 such that the total volume of the top reservoir 310 is now about 550 mL (third segment of FIG. 6 at Phase 2). At this stage, the microcontroller 340 has sensed the increase in volume of the top reservoir 310 from about 120 mL to about 550 mL so as to recognize that the patient has consumed food.

The microcontroller 340 recognizes that food intake has occurred at Phase 2, and, accordingly, sends a signal to the pump 330 to inflate the top reservoir 310 to about 700 mL, which represents the volume corresponding to this particular patient's induced satiety pressure level (Phase 3). The increase in volume and pressure of the top reservoir 310 is shown by the positive slope in FIGS. 6 and 7 from Phase 2 to Phase 3. In this example, the patient's induced satiety pressure level was empirically determined to be slightly less than about 12 mm Hg. Note that the microcontroller 340 has been programmed to not exceed the empirically determined discomfort pressure level of greater than about 12 mm Hg which corresponds to a top reservoir 310 volume of about 950 mL.

The volume of the reservoir 310 and the pressure of the reservoir 310 are held constant for a predetermined period of time, as shown at Phase 3. Preferably, the duration of Phase 3 is sufficient for all food contents to have exited the gastric lumen 360 and pass into the pylorus 381 and duodenum.

When peristalsis has occurred to pass the food contents from the gastric lumen 360 and into the pylorus 381, the pressure sensors may detect the decrease in volume of the gastric lumen 360 as a result of the peristalsis contractions. Alternatively, the microcontroller 340 may be programmed to activate the pump 330 to direct fluid from top reservoir 310 to bottom reservoir 320 after a predetermined time (e.g., 3 hours after food intake). Accordingly, the volume and the pressure of the top reservoir 310 decreases as shown in Phase 4, returning to its original volume and pressure as originally defined at Phase 1. In particular, fluid is directed from the top reservoir 310 to the bottom reservoir 320 through valve 350 such that the volume of the top reservoir 310 decreases and the volume of the bottom reservoir 320 proportionally increases so as to create the non-distended configuration shown in FIG. 2 and defined at Phase 4 of FIGS. 6 and 7. This cycle from Phase 1 to Phase 4 repeats in each instance that the gastric lumen 360 expands due to food intake. Although one intermediate plateau (i.e., Phase 2) was described in the example of FIGS. 6 and 7, more than one intermediate plateau may occur before the satiety induced pressure (Phase 3) is reached. It should be noted that the pressure of the reservoir 310 at Phases 1, 2, and 4 are identical. As can be seen, this pressure actuation embodiment as described in FIGS. 6 and 7 monitors and adjusts the volume of the reservoir 310 such that the reservoir 310 pressure is maintained at about 2 mm Hg above the MDP prior to ramping up to the satiety induced pressure level, both of which are empirically determined values for the particular patient prior to starting the procedure. The system has the ability to maintain a substantially constant pressure on the walls of the gastric lumen 360 (e.g., at Phase 2) before the satiety induced state at Phase 3 is achieved. This permits the patient to consume nutrients from food before the sensation of satiety is reached.

The reservoir described in the above embodiments may be any elastic, biocompatible, chemically inert material. For example, the reservoir may be formed from silicone, polyethylene, or polyurethane. The basic shape of the reservoir when fully inflated with fluid may be anatomically dependent on the elasticity of the material, the method of volume actuation, and the geometry of the gastric lumen.

Additionally, the reservoir may comprise a plurality of portions. Each of the plurality of portions may be interconnected by a pump and a microcontroller. The pump would be adapted to move fluid between each of the plurality portions in response to the one or more detected parameters by the microcontroller.

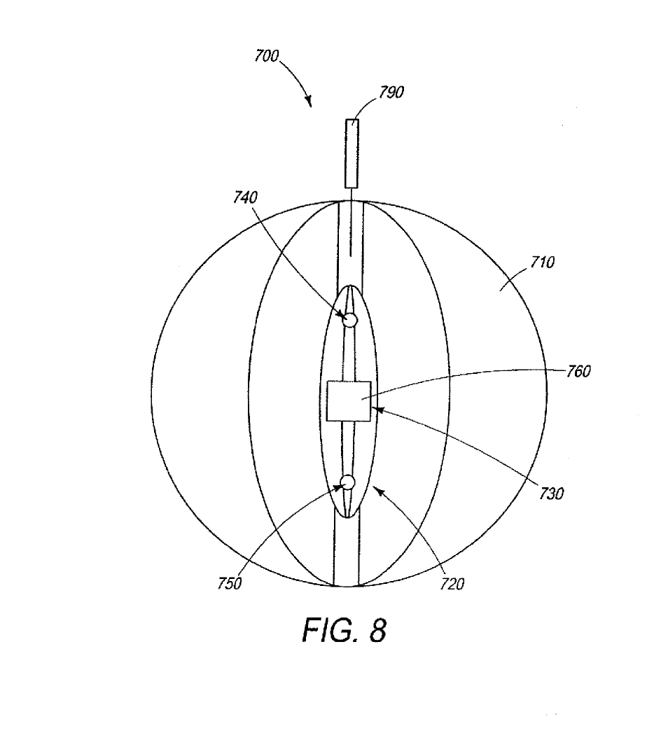

Several other types of dynamic volume actuation systems may be used to implement the above described pressure-controlled actuation. One example is shown in FIG. 8. FIG. 8 shows a pressure controlled actuation system 700. The system is sealed from the outside environment and comprises an expandable outer intragastric balloon 710, a semi-rigid inner chamber 720, and a pump 730 with a built-in microcontroller 760, an outtake valve 740, and an intake valve 750. A pressure transducer may be connected to the microcontroller 760. Compressed fluid (e.g., air) is housed within the inner chamber 720. When the gastric lumen expands during food intake such that the pressure against the outer balloon 710 decreases, the pressure transducer detects the lowering of pressure and sends a signal indicating such lowering of pressure to the microcontroller 760. The microcontroller 760 transmits a signal to the outtake valve 740 to open such that a predefined amount of air exits the inner chamber 720 and enters the outer balloon 710. The outer balloon 710 expands in response to the air entering the interior region of the outer balloon 710. The pressure of the outer balloon 710 against the walls of the gastric lumen increases to reestablish the pressure level as defined at Phase 2 FIGS. 6 and 7. The pressure transducer detects this pressure level and sends a signal indicating such pressure level to the microcontroller 760. The microcontroller 760 then transmits a signal to the outtake valve 740 to close. This process is repeated until the satiety induced pressure level (Phase 3 of FIGS. 6 and 7) is reached.

When the food contents have exited the pylorus, the walls of the gastric lumen contract by peristalsis. The pressure transducer senses that the outer balloon 710 is now exerting greater than the threshold satiety induced pressure level and accordingly transmits a signal indicating such a higher pressure level to the microcontroller 760. The microcontroller 760 sends a signal to cause the intake valve 750 to open and the pump 730 to activate. Opening of the intake valve 750 and activation of the pump 730 allows fluid from the outer balloon 710 to be suctioned back into the inner chamber 720 until the volume and pressure of the outer balloon 720 decreases and reaches the level defined at Phase 4 of FIGS. 6 and 7.

In order to reduce the pressurization of the inner chamber 720, an inflation catheter 790 may used to directly inject fluid into the outer balloon 71 0. This reduces the amount of fluid that needs to enter the interior of the outer balloon 710.

In the above-described embodiments, the microcontroller and pump may be powered by a variety of power sources known in the art for powering a monitoring system. In a preferred example, the microcontroller and pump are powered by batteries. The specific voltage requirement of the batteries is at least partially dependent upon the duration that the microcontroller and pump will be in use as well as the amperage load required to power the microcontroller and pump.

Although all of the above examples have described the process of distension occurring at the fundus of the stomach (i.e., the upper portion), distension may also occur at the antrum of the stomach (i.e., the lower portion) to induce satiety.

Other devices capable of dynamically changing volume are contemplated. As an example, a hydrogel may be used that is pH activated. The hydrogel may swell to a satiety inducing volume when the pH of the stomach is above about 3 (i.e., during food intake). The hydrogel may shrink when the pH of the stomach is below about 3 (i.e., between meals). The hydrogel may be fabricated from a prepolymer solution of poly(2-hydroxyethyl methacrylate) (HEMA)) gel. HEMA based hydrogels are known in the art to be sensitive to the pH of their aqueous environment, expanding at high pH and shrinking at low pH.

Additionally, the hydrogel may also be actuated to swell and shrink based on other stimuli, such as temperature. For example, the hydrogel may swell when the temperature of the gastric lumen decreases during food intake and shrink when the temperature of the gastric lumen increases between meals.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously. The designs described herein are intended to be exemplary only. The novel characteristics of the invention may be incorporated in other structural forms without departing from the spirit and scope of the invention.

1. An intragastric device for the treatment of obesity, the intragastric device comprising:
a reservoir comprising an elastic material that is configured to change volume while implanted within a gastric lumen, wherein the reservoir is actuated to change volume in response to one or more detected parameters, and further wherein the reservoir is adapted to distend one or more walls of the gastric lumen for a predetermined time.

2. The intragastric device according to claim 1, wherein the reservoir comprises an intragastric balloon.

3. The intragastric device according to claim 1, wherein the reservoir comprises fluid that is movable therewithin to change the volume.

4. The intragastric device according to claim 1, the reservoir further comprising a microcontroller, the microcontroller being in electrical communication with the reservoir, the microcontroller detecting the one or parameters, and the microcontroller regulating the actuation of the change in volume of the reservoir in response to the one or more parameters.

5. The intragastric device of claim 1, wherein the one or more detected parameters is a pressure being exerted on the reservoir.

6. The intragastric device of claim 1, wherein the one or more detected parameters is a pH of the gastric lumen.

7. The intragastric device of claim 1, the reservoir further comprising a first portion and a second portion, the first and the second portions being in fluid communication by a valve, the first and the second portions being transformable from a non-distended state to a distended state.

8. The intragastric device of claim 7, wherein the first and the second portions comprise fluid adapted to flow therebetween, the flow of the fluid between the first and the second portions adapted to alter the volume of the first and the second portions such that one of the first and the second portions transforms to the distended state in response to the one or detected parameters.

9. The intragastric device of claim 8, wherein a pump moves the fluid between the first and the second portions.

10. The intragastric device according to claim 1, the reservoir further comprising a plurality of portions, each of the plurality of portions interconnected by a pump and a microcontroller, the pump adapted to move fluid between each of the plurality portions in response to the one or more detected parameters by the microcontroller.

11. The intragastric device according to claim 1, wherein the reservoir is connected to an outside pump and a tube that connects the pump with the reservoir.

12. The intragastric device according to claim 11, wherein the tube extends through the stomach wall

13. The intragastric device according to claim 11, wherein the tube extends along the esophagus.

14. An intragastric device for the treatment of obesity, the intragastric device comprising:
an expandable reservoir that is configured to change volume while implanted in a gastric lumen, the reservoir being actuated by a pressure controller to change volume in response to a pressure change of the reservoir against one or more walls of the gastric lumen, the reservoir adapted to distend the one or more walls of the gastric lumen for a predetermined time to trigger a sensation of satiety.

15. The intragastric device according to claim 14, the reservoir further comprising an outer shell, and a chamber located within the outer shell, the chamber comprising gas that is flowable between the chamber and the outer shell, the chamber further comprising an intake valve and a pump for redirecting the gas into the chamber.

16. The intragastric device according to claim 14, wherein the pressure controller comprises a pressure transducer and a microcontroller.

17. A method of treatment of obesity, the method comprising the steps of:
(a) introducing a reservoir into a gastric lumen, the reservoir having a first volume,
(b) detecting a parameter within the gastric lumen, the parameter being indicative of expansion of the gastric lumen; and
(c) actuating the reservoir based on the detected parameter such that the reservoir changes from the first volume to a second volume, the second volume being larger than the first volume.

18. The method of claim 17, further comprising the step of:
(d) engaging a wall of the gastric lumen to distend the wall of the gastric lumen for a predetermined time.

19. The method of claim 17, wherein the step of detecting the parameter comprises measuring a first pressure exerted against the reservoir by the wall of the gastric lumen.

20. The method of claim 18, wherein the step of engaging the wall to distend the wall of the gastric lumen comprises the reservoir generating a second pressure against the wall, the first pressure being about equal to the second pressure.

21. The method of claim 20, further comprising the step of:
(e) actuating the reservoir based on the detected parameter such that the reservoir changes from the second volume to a third volume, the third volume being larger than the second volume, and the third volume pressure-inducing a sensation of satiety.

22. The method of claim 21, further comprising the step of:
(f) actuating the reservoir based on the detected parameter such that the reservoir changes from the third volume to the first volume in response to peristalsis contraction of the gastric lumen.
